(19) United States
(12) Patent Application Publication
(10) Pub. No.: US 2024/0293005 A1
(43) Pub. Date: Sep. 5, 2024

(54) ENDOSCOPE DISTAL END, ENDOSCOPE, AND ENDOSCOPE SYSTEM

(71) Applicant: OLYMPUS MEDICAL SYSTEMS CORP., Tokyo (JP)

(72) Inventor: Takahiro SUWA, Tokyo (JP)

(73) Assignee: OLYMPUS MEDICAL SYSTEMS CORP., Tokyo (JP)

(21) Appl. No.: 18/589,795

(22) Filed: Feb. 28, 2024

Related U.S. Application Data
(60) Provisional application No. 63/449,648, filed on Mar. 3, 2023.

(51) Int. Cl. A61B 1/00; A61B 1/05; H04N 23/50

(52) U.S. Cl. CPC A61B 1/00098; A61B 1/00045; A61B 1/00193; A61B 1/05; H04N 23/555

(57) ABSTRACT

A distal end portion for use with an endoscope includes: an imager configured to capture an image of a predetermined three-dimensional area; and a raising base configured to be rotatable in a predetermined rotational range, the raising base configured to abut against a treatment tool to adjust a direction in which the treatment tool protrudes. The imager is arranged such that a portion of the raising base is in the predetermined three-dimensional area when the raising base is positioned anywhere within the predetermined rotation range.

### CROSS-REFERENCE TO RELATED APPLICATION

This application is based on and claims priority under 35 U.S.C. § 119 to U.S. Provisional Application No. 63/449,648, filed Mar. 3, 2023, the entire contents of which are incorporated herein by reference.

### BACKGROUND

The present disclosure relates to endoscope distal ends, endoscopes, and endoscope systems.

An endoscope for observation of the interior of a subject, such as a human, has been known, the endoscope having a flexible and elongated insertion portion to be inserted into the subject (see, for example, Japanese Patent Application Laid-open No. 2021-007805).

An endoscope described in Japanese Patent Application Laid-open No. 2021-007805 has a treatment tool raising stand and an imager that are described below and provided at a distal end of an insertion portion thereof.

The treatment tool raising stand is set, by rotating, in a raised state where the treatment tool raising stand has been raised or a laid state where the treatment tool raising stand has been laid, with respect to the longitudinal direction of the insertion portion, and adjusts, by abutting against a treatment tool that has been inserted through the insertion portion, the direction in which the treatment tool protrudes from the insertion portion.

The imager performs imaging for a subject image and outputs an image signal corresponding to the imaging. The image signal is input to a video processor outside the endoscope. The video processor generates an endoscopic image by processing the image signal and causes a display to display the endoscopic image.

### SUMMARY

In some embodiments, a distal end portion for use with an endoscope includes: an imager configured to capture an image of a predetermined three-dimensional area; and a raising base configured to be rotatable in a predetermined rotational range, the raising base configured to abut against a treatment tool to adjust a direction in which the treatment tool protrudes. The imager is arranged such that a portion of the raising base is in the predetermined three-dimensional area when the raising base is positioned anywhere within the predetermined rotation range.

In some embodiments, an endoscope includes: an insertion portion that has a distal end portion provided at a distal end of the insertion portion, the insertion portion being configured to be inserted into a subject. The distal end portion includes: an imager configured to capture an image of a predetermined three-dimensional area; and a raising base configured to be rotatable in a predetermined rotational range, the raising base configured to abut against a treatment tool to adjust a direction in which the treatment tool protrudes, and the imager is arranged such that a portion of the raising base is in the predetermined three-dimensional area when the raising base is positioned anywhere within the predetermined rotation range.

In some embodiments, an endoscope system includes: an insertion portion configured to be inserted into a subject and to capture an image of an interior of the subject to output an image signal; an image processor configured to process the image signal to generate an endoscopic image; and a display configured to display the endoscopic image. The insertion portion has a distal end portion provided at a distal end of the insertion portion, the distal end portion includes: an imager configured to capture the image of the interior of the subject; and a raising base configured to be rotatable in a predetermined rotational range, the raising base configured to abut against a treatment tool to adjust a direction in which the treatment tool protrudes, and the imager is arranged such that a portion of the raising base is captured in the endoscopic image when the raising base is positioned anywhere within the predetermined rotation range.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an endoscope system according to an embodiment.

FIG. 2 is a diagram illustrating a configuration of an endoscope distal end.

FIG. 3 is a diagram illustrating the configuration of the endoscope distal end.

FIG. 4 is a diagram illustrating an observation field of an imager.

FIG. 5 is a diagram illustrating a first example of a relation between a raising stand trajectory relative to the observation field and the raising stand trajectory on an endoscopic image.

FIG. 6 is a diagram illustrating the first example of the relation between the raising stand trajectory relative to the observation field and the raising stand trajectory on the endoscopic image.

FIG. 7 is a diagram illustrating a second example of the relation between the raising stand trajectory relative to the observation field and the raising stand trajectory on the endoscopic image.

FIG. 8 is a diagram illustrating the second example of the relation between the raising stand trajectory relative to the observation field and the raising stand trajectory on the endoscopic image.

FIG. 9 is a diagram illustrating a third example of the relation between the raising stand trajectory relative to the observation field and the raising stand trajectory on the endoscopic image.

FIG. 10 is a diagram illustrating the third example of the relation between the raising stand trajectory relative to the observation field and the raising stand trajectory on the endoscopic image.

### DETAILED DESCRIPTION

Modes for implementing the disclosure (hereinafter, embodiments) will be described hereinafter by reference to the drawings. The disclosure is not limited by the embodiments described hereinafter. Furthermore, the same reference sign is assigned to portions that are the same, throughout the drawings.

### Schematic Configuration of Endoscope System

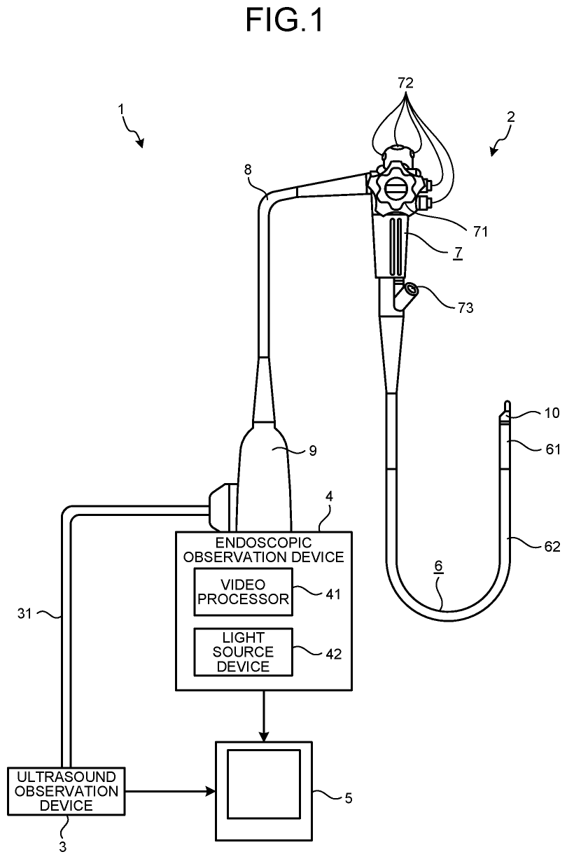

FIG. 1 is a diagram illustrating an endoscope system 1 according to an embodiment.

The endoscope system 1 is a system for ultrasound diagnosis of the interior of a subject, such as a human, by use of an ultrasound endoscope. This endoscope system 1 includes, as illustrated in FIG. 1, an ultrasound endoscope 2, an ultrasound observation device 3, an endoscopic observation device 4, and a display 5.

The ultrasound endoscope 2 corresponds to an endoscope. Part of this ultrasound endoscope 2 is capable of being inserted into a subject, and the ultrasound endoscope 2 has a function of transmitting ultrasound pulses to a body wall inside the subject, receiving ultrasound echoes reflected by the subject, and outputting an echo signal and a function of outputting an image signal by capturing an image of the interior of the subject.

A detailed configuration of the ultrasound endoscope 2 will be described in a later section, “Configuration of Ultrasound Endoscope”.

In this embodiment, the ultrasound endoscope 2 is adopted as the endoscope, but without being limited to this embodiment, an endoscope, such as a duodenoscope, not having an ultrasound probe 11 may be adopted instead.

The ultrasound observation device 3 is electrically connected to the ultrasound endoscope 2 via an ultrasound cable 31 (FIG. 1), and outputs a pulse signal to the ultrasound endoscope 2 and inputs an echo signal from the ultrasound endoscope 2 via the ultrasound cable 31. The ultrasound observation device 3 generates an ultrasound image by performing predetermined processing of the echo signal.

A later described endoscope connector 9 (FIG. 1) of the ultrasound endoscope 2 is detachably connected to the endoscopic observation device 4. The endoscopic observation device 4 includes, as illustrated in FIG. 1, a video processor 41 and a light source device 42.

The video processor 41 corresponds to an image processor. The video processor 41 receives an image signal from the ultrasound endoscope 2 via the endoscope connector 9. The video processor 41 generates an endoscopic image by performing predetermined processing of the image signal.

The light source device 42 supplies illumination light for illuminating the interior of a subject, to the ultrasound endoscope 2 via the endoscope connector 9.

The display 5 is a display using liquid crystal or organic electro luminescence (EL), and displays thereon, for example, an ultrasound image generated by the ultrasound observation device 3 or an endoscopic image generated by the endoscopic observation device 4.

### Configuration of Ultrasound Endoscope

The ultrasound endoscope 2 includes, as illustrated in FIG. 1, an insertion portion 6, an operating portion 7, a universal cord 8, and the endoscope connector 9.

“Distal” referred to hereinafter means being near, toward, or in the direction of a distal end of the insertion portion 6 (forward in the direction of insertion into a subject). Furthermore, “proximal” referred to hereinafter means being near, toward, or in the direction of an end away from the distal end of the insertion portion 6.

The insertion portion 6 is a portion to be inserted into a subject. The insertion portion 6 includes, as illustrated in FIG. 1, an endoscope distal end 10, a bending portion 61, and a flexible tube 62.

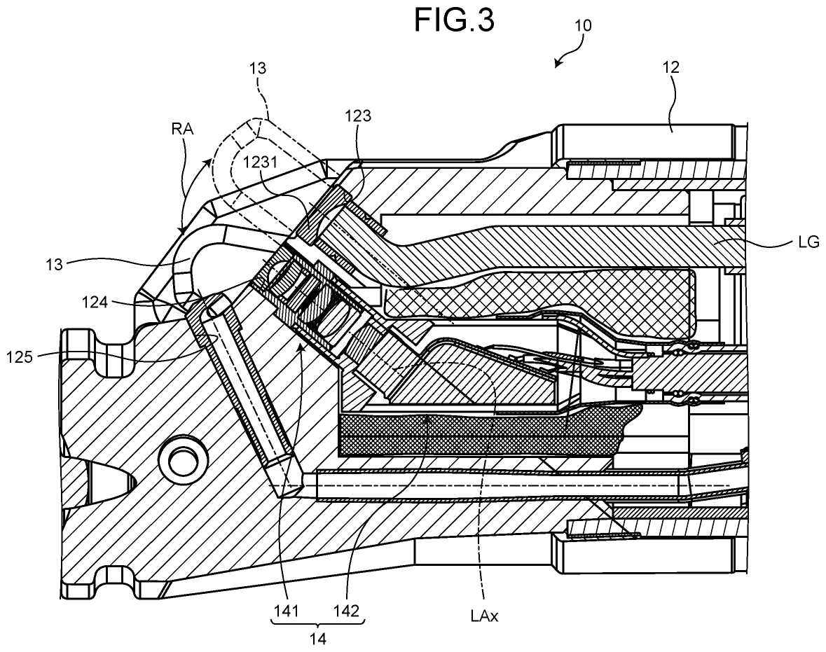

Inside the insertion portion 6, the operating portion 7, the universal cord 8, and the endoscope connector 9: a light guide LG (see FIG. 3) that transmits illumination light supplied from the light source device 42, a transducer cable (not illustrated in the drawings) that transmits the above described pulse signal and echo signal, and a signal cable (not illustrated in the drawings) that transmits an image signal have been laid; and a duct line (not illustrated in the drawings) for circulating fluid has been provided.

The endoscope distal end 10 (distal end portion) is provided at the distal end of the insertion portion 6.

A detailed configuration of the endoscope distal end 10 will be described in a later section, “Configuration of Endoscope Distal End”.

The bending portion 61 is connected near a proximal end of the endoscope distal end 10 and is bendable.

The flexible tube 62 is connected near a proximal end of the bending portion 61 and has flexibility.

The operating portion 7 is a portion that is connected near the proximal end of the insertion portion 6 and receives various operations from a medical doctor, for example. This operating portion 7 includes, as illustrated in FIG. 1, a bending knob 71 for operating the bending portion 61 to bend the bending portion 61, and plural operating portions 72 for performing various operations.

Furthermore, the operating portion 7 has, provided therein, a treatment tool insertion port 73 that is in communication with a tube (not illustrated in the drawings) provided inside the bending portion 61 and the flexible tube 62, the treatment tool insertion port 73 being for insertion of a treatment tool (not illustrated in the drawings) into the tube.

The universal cord 8 is a cord that extends from the operating portion 7 and that has, arranged therein: the light guide LG, the above mentioned transducer cable (not illustrated in the drawings); the above mentioned signal cable (not illustrated in the drawings); and the above mentioned duct line (not illustrated in the drawings).

The endoscope connector 9 is provided at an end portion of the universal cord 8. The ultrasound cable 31 is connected to the endoscope connector 9 and the endoscope connector 9 is connected to the video processor 41 and the light source device 42 by being plugged into the endoscopic observation device 4.

### Configuration of Endoscope Distal End

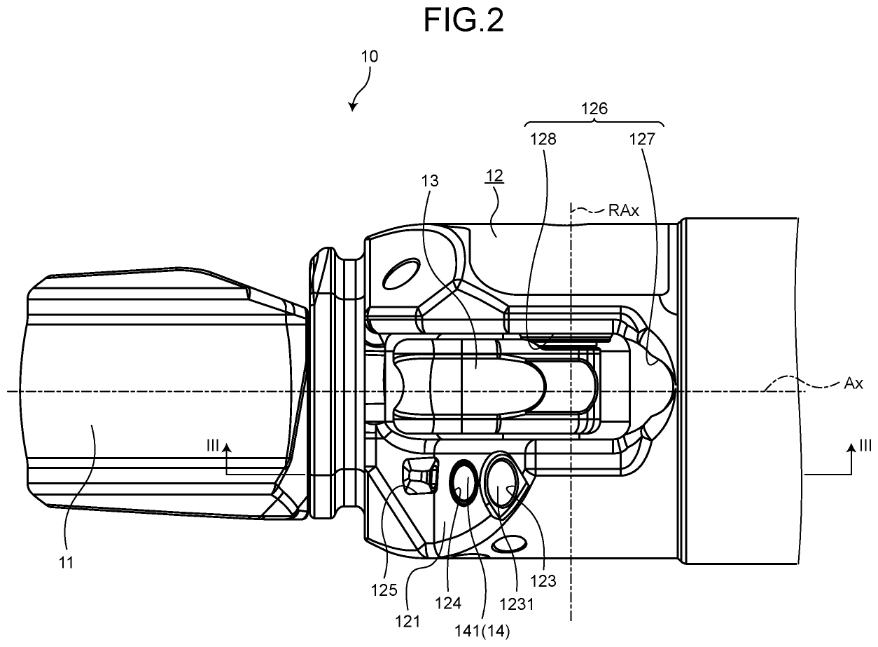

FIG. 2 and FIG. 3 are diagrams illustrating a configuration of the endoscope distal end 10. Specifically, FIG. 2 is a diagram of the endoscope distal end 10 as viewed from the left in FIG. 1. FIG. 3 is a sectional view for a line III-III illustrated in FIG. 2.

The endoscope distal end 10 includes, as illustrated in FIG. 2 and FIG. 3, the ultrasound probe 11, a distal end component 12, and a treatment tool raising stand 13 (raising base).

The ultrasound probe 11 is a convex ultrasound probe and has plural ultrasound transducers (not illustrated in the drawings) regularly arranged in a state of forming a convex circular arc. The ultrasound probe 11 adopted is not necessarily a convex ultrasound probe, and a radial ultrasound probe may be adopted instead.

Each of the ultrasound transducers includes an acoustic lens, a piezoelectric element, and a matching layer, and acquires ultrasound echoes contributing to an ultrasound tomographic image of the interior of a subject, the interior being more inside than a body wall of the subject.

The ultrasound probe 11 converts a pulse signal input from the ultrasound observation device 3 via the ultrasound cable 31 and the above mentioned transducer cable into ultrasound pulses and transmits the ultrasound pulses into a subject. Furthermore, the ultrasound probe 11 converts ultrasound echoes reflected inside the subject, into an electric echo signal, and outputs the electric echo signal via the above mentioned transducer cable and ultrasound cable 31, to the ultrasound observation device 3.

The ultrasound probe 11 described above is arranged, as illustrated in FIG. 2, more distally (lower in FIG. 2) than the treatment tool raising stand 13.

The distal end component 12 is a rigid portion made of, for example, a resin material, and has an approximately cylindrical shape extending along a central axis Ax of the insertion portion 6.

This distal end component 12 has an inclined surface 121 (FIG. 2) provided on an outer peripheral surface near a distal end of the distal end component 12, the inclined surface 121 making the distal end component 12 tapered toward the distal end of the distal end component 12.

The distal end component 12 has, provided therein, for example: an attachment hole (not illustrated in the drawings) penetrating the distal end component 12 from a proximal end to the distal end of the distal end component 12; and an illumination hole 123 (FIG. 2 and FIG. 3), an imaging hole 124 (FIG. 2 and FIG. 3), a gas and water feeding hole 125 (FIG. 2 and FIG. 3), and a treatment tool channel 126 (FIG. 2) that each penetrate the distal end component 12 from the proximal end to the inclined surface 121.

The attachment hole (not illustrated in the drawings) is a hole where the ultrasound probe 11 is to be attached. The above mentioned transducer cable electrically connected to the ultrasound probe 11 is inserted inside the attachment hole.

The illumination hole 123 has, arranged therein, an output end of the light guide LG and an illumination lens 1231 that illuminates the interior of a subject with illumination light output from the output end of the light guide LG, as illustrated in FIG. 3.

The imaging hole 124 has, provided therein, an imager 14.

The imager 14 includes: an objective optical system 141 (FIG. 3) that condenses light (subject image) that has been emitted into a subject and reflected in the subject; and an imaging element 142 (FIG. 3) that captures the subject image condensed by the objective optical system 141. An image signal captured by the imaging element 142 is transmitted to the endoscopic observation device 4 (video processor 41) via the above mentioned signal cable (not illustrated in the drawings).

The gas and water feeding hole 125 is part of the above mentioned duct line (not illustrated in the drawings) and is a hole for cleaning an outer surface of the objective optical system 141 by allowing gas or water to be fed to the imaging hole 124.

The treatment tool channel 126 is a passage that lets a treatment tool (not illustrated in the drawings) protrude outside, the treatment tool having been inserted through the above mentioned tube (not illustrated in the drawings) inside the insertion portion 6 from the treatment tool insertion port 73. This treatment tool channel 126 includes, as illustrated in FIG. 2, a treatment tool insertion hole 127 and a storage groove 128.

The treatment tool insertion hole 127 is a portion extending from the proximal end of the distal end component 12 toward the distal end of the distal end component 12, the portion being where a treatment tool (not illustrated in the drawings) is to be inserted through.

The storage groove 128 is a groove that is in communication with the treatment tool insertion hole 127 and that extends toward the distal end from the treatment tool insertion hole 127 along the central axis Ax.

The treatment tool raising stand 13 is stored inside the storage groove 128 such that the treatment tool raising stand 13 is rotatable about a rotation axis RAx (FIG. 2) according to a user operation of the operating portion 7 by, for example, a medical doctor. The treatment tool raising stand 13 is set, by rotating about the rotation axis RAx, in a raised state where the treatment tool raising stand 13 has been raised or a laid state where the treatment tool raising stand 13 has been laid, with respect to the central axis Ax, and the treatment tool raising stand 13 adjusts, by abutting against a treatment tool (not illustrated in the drawings) that has been inserted through the treatment tool insertion hole 127 into the storage groove 128, the direction in which the treatment tool protrudes. In FIG. 3, the treatment tool raising stand 13 that has been set in the raised state is illustrated with dashed and dotted lines and the treatment tool raising stand 13 that has been set in the laid state is illustrated with solid lines.

A distal end of the imager 14 described above is arranged, as illustrated in FIG. 2, more proximally (up in FIG. 2) than a distal end of the treatment tool raising stand 13.

### Observation Field of Imager

An observation field of the imager 14 described above will be described next.

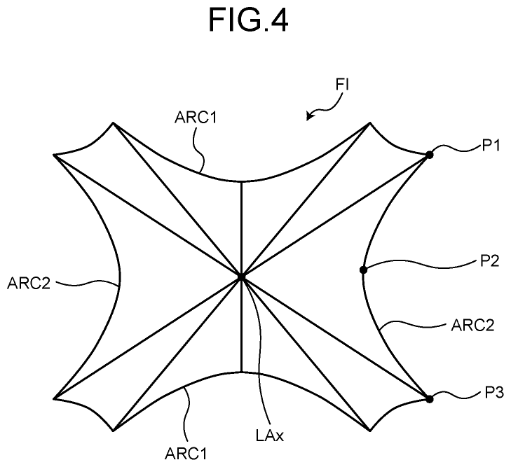

FIG. 4 is a diagram illustrating an observation field FI of the imager 14. Specifically, FIG. 4 is a diagram of the observation field FI of the imager 14, as viewed along an optical axis LAx of the objective optical system 141 from the center of the objective optical system 141 and is a section cut along a plane orthogonal to the optical axis LAx, the section being deeper inside the plane of paper of FIG. 4.

The video processor 41 generates an endoscopic image F1 (see FIG. 6, FIG. 8, and FIG. 10) by performing predetermined processing of an image signal output from the imager 14, the endoscopic image F1 having, in its entire square area: a masked area MAr corresponding to its four corners that have been masked (blacked out); and a subject captured in only an image area FAr that is other than the masked area MAr and octagonal. The observation field FI forming the image area FAr that is captured by the imager 14 and is octagonal has the shape illustrated in FIG. 4. The observation field FI corresponds to a predetermined three-dimensional area.

The observation field FI has a shape vertically symmetric about a horizontal plane including the optical axis LAx and also a shape horizontally symmetric about a vertical plane including the optical axis LAx. More specifically, a cross section of the observation field FI when the observation field FI is cut along a plane orthogonal to the optical axis LAx includes a pair of circular arcs ARC1 vertically faced each other with the optical axis LAx interposed between the pair of circular arcs ARC1 and a pair of circular arcs ARC2 horizontally faced each other with the optical axis LAx interposed between the pair of circular arcs ARC2.

The circular arcs ARC1 and ARC2 are circular arcs recessed toward the optical axis LAx. The uppermost position of the circular arcs ARC2 in FIG. 4 corresponds to a first position P1. Furthermore, the lowermost position of the circular arcs ARC2 in FIG. 4 corresponds to a third position P3. Furthermore, an intermediate position between the first and third positions P1 and P3 on the circular arcs ARC2 corresponds to a second position P2.

A rotational range between the raised state and the laid state of the treatment tool raising stand 13 corresponds to a predetermined rotational range RA (FIG. 3). Furthermore, a trajectory of the distal end of the treatment tool raising stand 13 that has rotated in the rotational range RA will hereinafter be referred to as a raising stand trajectory TR (FIG. 3).

The treatment tool raising stand 13 is arranged such that the raising stand trajectory TR crosses the observation field FI. In other words, the imager 14 is set to be positioned such that the distal end of the treatment tool raising stand 13 is in the observation field FI no matter where in the rotational range RA the treatment tool raising stand 13 is positioned. No matter where in the rotational range RA the treatment tool raising stand 13 is positioned, the distal end of the treatment tool raising stand 13 is thereby captured in the image area FAr in the endoscopic image F1 and displayed on the display 5.

### Relation Between Raising Stand Trajectory Relative to Observation Field and Raising Stand Trajectory on Endoscopic Image

A relation between the raising stand trajectory TR relative to the observation field FI and the raising stand trajectory TR on the endoscopic image F1 will be described next.

A first example to a third example of this relation will hereinafter be described in order.

### First Example

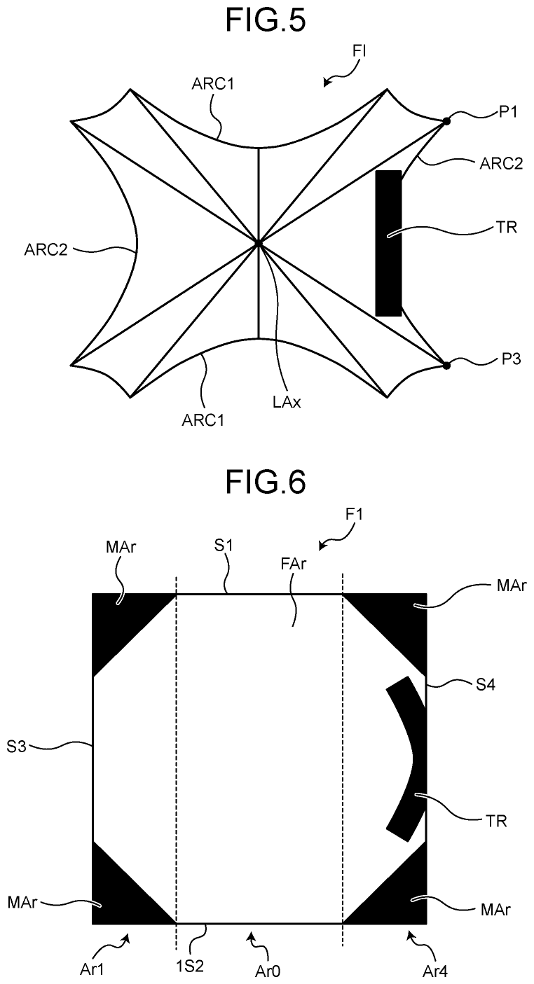

FIG. 5 and FIG. 6 are diagrams illustrating the first example of the relation between the raising stand trajectory TR relative to the observation field FI and the raising stand trajectory TR on the endoscopic image F1. Specifically, FIG. 5 is a diagram corresponding to FIG. 4 and is a diagram illustrating the raising stand trajectory TR relative to the observation field FI. FIG. 6 is a diagram illustrating the raising stand trajectory TR on the endoscopic image F1.

The upper side of the endoscopic image F1 will hereinafter be referred to as an upper side S1, the lower side as a lower side S2, the left side as a left side S3, and the right side as a right side S4. Furthermore, in FIG. 6, a first area Ar1 is an area along the left side S3, the area being one of four areas resulting from equal division of the endoscopic image F1 into four along the upper side S1. Furthermore, a fourth area Ar4 is an area along the right side S4, the area being one of the four areas. Furthermore, a central area ArO is an area positioned between the first and fourth areas Ar1 and Ar4, the area being in the four areas, and corresponds to a second and third areas. The same applies to FIG. 8 and FIG. 10 described below.

As illustrated in FIG. 5, in a case supposed herein, the raising stand trajectory TR vertically crosses a right area of the observation field FI in FIG. 5 such that the raising stand trajectory TR is parallel to a linear virtual line joining the first and third positions P1 and P3. In this case, as illustrated in FIG. 6, the raising stand trajectory TR on the endoscopic image F1 is a circular arc that is positioned in the image area FAr in the fourth area Ar4, protrudes rightward in FIG. 6, and is vertically symmetrical about a virtual line parallel to the upper side S1. That is, the raising stand trajectory TR is captured in the endoscopic image F1 along the right side S4.

The uppermost position of the raising stand trajectory TR in FIG. 5 and FIG. 6 is the position in the raised state. Furthermore, the lowermost position of the raising stand trajectory TR in FIG. 5 and FIG. 6 is the position in the laid state.

### Second Example

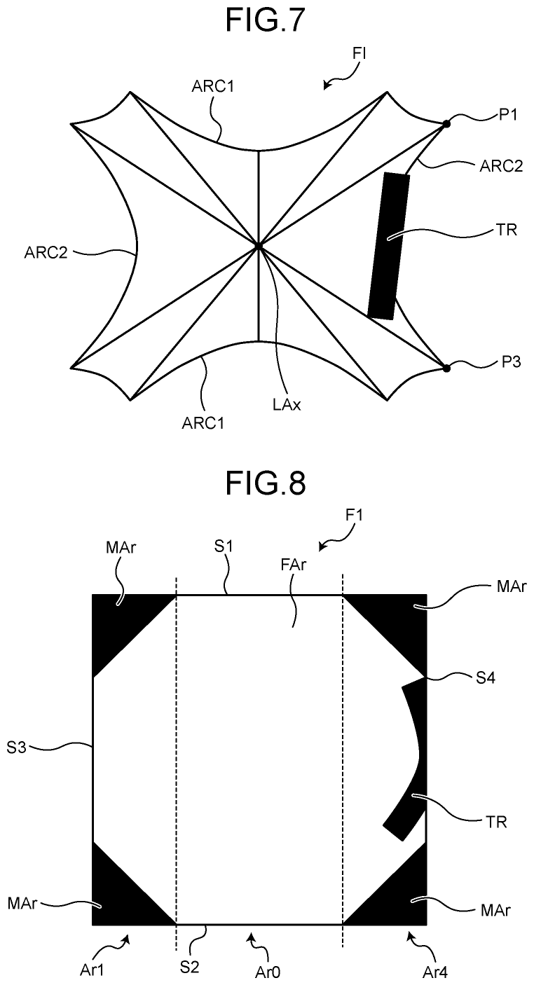

FIG. 7 and FIG. 8 are diagrams illustrating the second example of the relation between the raising stand trajectory TR relative to the observation field FI and the raising stand trajectory TR on the endoscopic image F1. Specifically, FIG. 7 is a diagram corresponding to FIG. 4 and is a diagram illustrating the raising stand trajectory TR relative to the observation field FI. FIG. 8 is a diagram illustrating the raising stand trajectory TR on the endoscopic image F1.

As illustrated in FIG. 7, in a case supposed herein, the raising stand trajectory TR vertically crosses a right area of the observation field FI in FIG. 7 along a straight line slanted to extend leftward downward in the right area, instead of being parallel to a linear virtual line joining the first and third positions P1 and P3. In this case, as illustrated in FIG. 8, the raising stand trajectory TR on the endoscopic image F1 is a circular arc that is positioned in the image area FAr in the fourth area Ar4, protrudes rightward in FIG. 8, and is not vertically symmetrical about a virtual line parallel to the upper side S1. More specifically, the raising stand trajectory TR on the endoscopic image F1 is a circular arc in which a position closest to the lower side S2 on the raising stand trajectory TR is closer to a center C (FIG. 8) of the endoscopic image F1 than a position closest to the upper side S1 on the raising stand trajectory TR. That is, the raising stand trajectory TR is captured in the endoscopic image F1 along the right side S4.

The uppermost position of the raising stand trajectory TR in FIG. 7 and FIG. 8 is the position in the raised state. Furthermore, the lowermost position of the raising stand trajectory TR in FIG. 7 and FIG. 8 is the position in the laid state.

### Third Example

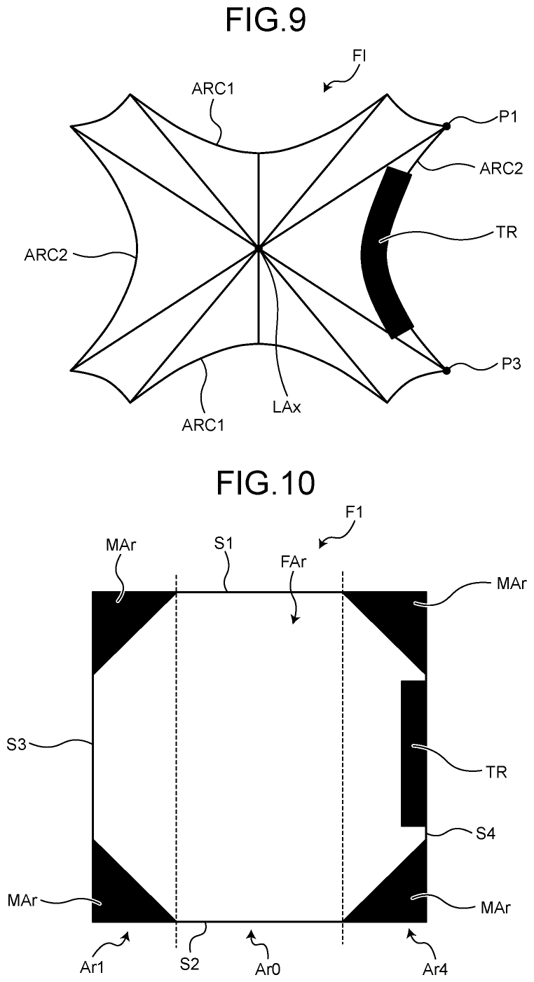

FIG. 9 and FIG. 10 are diagrams illustrating the third example of the relation between the raising stand trajectory TR relative to the observation field FI and the raising stand trajectory TR on the endoscopic image F1. Specifically, FIG. 9 is a diagram corresponding to FIG. 4 and is a diagram illustrating the raising stand trajectory TR relative to the observation field FI. FIG. 10 is a diagram illustrating the raising stand trajectory TR on the endoscopic image F1.

As illustrated in FIG. 9, in a case supposed herein, the raising stand trajectory TR crosses the observation field FI in a circular arc shape along the right circular arc ARC2 in the observation field FI in FIG. 9. In this case, the raising stand trajectory TR on the endoscopic image F1 is, as illustrated in FIG. 10, a straight line that is positioned in the image area FAr in the fourth area Ar4 and that is parallel to the right side S4. That is, the raising stand trajectory TR is captured in the endoscopic image F1 along the right side S4.

The uppermost position of the raising stand trajectory TR in FIG. 9 and FIG. 10 is the position in the raised state. Furthermore, the lowermost position of the raising stand trajectory TR in FIG. 9 and FIG. 10 is the position in the laid state.

The above described embodiment has the following effects.

The treatment tool raising stand 13 in the endoscope distal end 10 according to the embodiment is arranged such that the raising stand trajectory TR crosses the observation field FI. In other words, the imager 14 is set to be positioned such that the distal end of the treatment tool raising stand 13 is in the observation field FI no matter where in the rotational range RA the treatment tool raising stand 13 is positioned. No matter where in the rotational range RA the treatment tool raising stand 13 is positioned, the distal end of the treatment tool raising stand 13 is thereby captured in the image area FAr in the endoscopic image F1 and displayed on the display 5.

Therefore, the endoscope distal end 10 according to the embodiment enables the position and posture of a treatment tool to be known from the endoscopic image F1 displayed on the display 5 and enables treatment to be performed smoothly, the treatment using the treatment tool. That is, the usability is able to be improved.

### Other Embodiments

Modes for implementing the disclosure have been described thus far, but the disclosure should not be limited only to the embodiments described above.

In the above described embodiment, the endoscope system 1 has both the function of generating an ultrasound image and the function of generating an endoscopic image, but not being limited to this embodiment, the endoscope system 1 may be configured to have only one of these functions.

In the above described embodiment, the endoscope system 1 may be an endoscope system for observation of the interior of a subject, such as a mechanical structure in the industrial field, without being limited to that in the medical field.

In the above described embodiment, any other positional relation between the imager 14 and the treatment tool raising stand 13 may be adopted as long as the distal end of the imager 14 is arranged more proximally than the distal end of the treatment tool raising stand 13. That is, in the above described embodiment, the imager 14 is arranged more rightward than the treatment tool raising stand 13 as viewed from the distal end of the insertion portion 6, as illustrated in FIG. 2, but without being limited to this embodiment, the imager 14 may be arranged at another position, such as a leftward position.

An endoscope distal end, an endoscope, and an endoscope system, according to the disclosure, enable improvement in usability thereof.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A distal end portion for use with an endoscope, comprising:
an imager configured to capture an image of a predetermined three-dimensional area; and
a raising base configured to be rotatable in a predetermined rotational range, the raising base configured to abut against a treatment tool to adjust a direction in which the treatment tool protrudes, wherein
the imager is arranged such that a portion of the raising base is in the predetermined three-dimensional area when the raising base is positioned anywhere within the predetermined rotation range.

2. The distal end portion according to claim 1, wherein the portion of the raising base is a distal end of the raising base.

3. The distal end portion according to claim 2, wherein a distal end of the imager is arranged proximally relative to the distal end of the raising base.

4. The distal end portion according to claim 3, wherein a trajectory of the distal end of the raising base when the raising base is rotated in the predetermined rotational range crosses the predetermined three-dimensional area.

5. The distal end portion according to claim 4, wherein
a cross section of the predetermined three-dimensional area being cut along a plane orthogonal to an optical axis of the imager includes a circular arc passing through a first position, a second position, and a third position in order, and
the second position is closer to the optical axis than each of the first position and the third position.

6. The distal end portion according to claim 5, wherein
the cross section of the predetermined three-dimensional area includes two circular arcs facing each other with the optical axis interposed, and
the circular arc is one of the two circular arcs.

7. An endoscope comprising:
an insertion portion that has a distal end portion provided at a distal end of the insertion portion, the insertion portion being configured to be inserted into a subject, wherein
the distal end portion comprises:
an imager configured to capture an image of a predetermined three-dimensional area; and
a raising base configured to be rotatable in a predetermined rotational range, the raising base configured to abut against a treatment tool to adjust a direction in which the treatment tool protrudes, and
the imager is arranged such that a portion of the raising base is in the predetermined three-dimensional area when the raising base is positioned anywhere within the predetermined rotation range.

8. An endoscope system comprising:
an insertion portion configured to be inserted into a subject and to capture an image of an interior of the subject to output an image signal;
an image processor configured to process the image signal to generate an endoscopic image; and
a display configured to display the endoscopic image, wherein
the insertion portion has a distal end portion provided at a distal end of the insertion portion,
the distal end portion comprises:
an imager configured to capture the image of the interior of the subject; and
a raising base configured to be rotatable in a predetermined rotational range, the raising base configured to abut against a treatment tool to adjust a direction in which the treatment tool protrudes, and
the imager is arranged such that a portion of the raising base is captured in the endoscopic image when the raising base is positioned anywhere within the predetermined rotation range.

9. The endoscope system according to claim 8, wherein the portion of the raising base is a distal end of the raising base.

10. The endoscope system according to claim 9, wherein a distal end of the imager is arranged proximally relative to a direction in which the insertion portion is inserted, and proximally relative to the distal end of the raising base.

11. The endoscope system according to claim 10, wherein a trajectory of the distal end of the raising base when the raising base is rotated in the predetermined rotational range is captured in the endoscopic image.

12. The endoscope system according to claim 11, wherein
the endoscopic image is an image that has a shape having an upper side, a lower side, a left side, and a right side, and
the trajectory is captured in the endoscopic image along the left side or the right side.

13. The endoscope system according to claim 12, wherein
when the endoscopic image is equally divided into four areas that are a first area, a second area, a third area, and a fourth area along the upper side or the lower side, the trajectory is captured in the first area along the left side or in the fourth area along the right side.

14. The endoscope system according to claim 13, wherein
a position closest to the lower side on the trajectory is captured at a position closer to a center of the endoscopic image than a position closest to the upper side on the trajectory.
